SEQUENCE LISTING (12 sequences)

SEQ ID NO: 1 - protein, 10 aa, Artificial
  Fragment of Spike Protein
YEVFEKVHVQ

SEQ ID NO: 2 - protein, 1376 aa, Artificial
  Fragment of PEDV spike protein
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDISVILAEGSIVLHTALGTNFSFVCSNSSDPHLATFAIPLGAIQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTEYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQP

SEQ ID NO: 3 - protein, 1379 aa, Artificial
  Mutated Spike protein of PEDV
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDISVILAEGSIVLHTALGTNFSFVCSNSSDPHLATFAIPLGAIQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTEYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLKLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPLAL

SEQ ID NO: 4 - protein, 5 aa, Artificial
  fragment of mutated Spike protein of PEDV
QPLAL

SEQ ID NO: 5 - protein, 27 aa, Artificial
  Peptide expressed by newly formed ORF
MRSLLFLILRHFLSSMVKQLIMTANPL

SEQ ID NO: 6 - DNA, 370 bp, Artificial
  Fragment deleted from ORF-2/ORF-3 of PEDV
acgaagtttttgaaaaggtccacgtgcagtgatgtttcttggactttttcaatacacgattgacacagttttcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcc

SEQ ID NO: 7 - DNA, 26 bp, Artificial
  Fragment deleted in ORF-3 of PEDV
gttggcattactaatgctgcattcta

SEQ ID NO: 8 - protein, 1386 aa, Porcine Pidemic Diarrea virus
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDISVILAEGSIVLHTALGTNFSFVCSNSSDPHLATFAIPLGAIQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTEYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 9 - DNA, 84 bp, Artificial
  New Orf created as a result of mutations in PEDV
atgcgctctttattatttttaatactacgacactttctttcctcaatggtaaagcagcttattatgacggcaaatccattgtga

SEQ ID NO: 10 - DNA, 27642 bp, Artificial
  Full genome of mutated PEDV virus
acttaaagagattttctatctatggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccatcctccggttccgtctgggggttgcgtggataactagttccgtctagtttgaaaccagtaactgtcggctatggctagcaaccaagtcacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccagaaacatttgtggttggctgttattttctaactgtaattacttcctcgaagagttagagctcacttttggtcgtcgtggtggtaacatcgtgccagttgaccaatacatgtgtggcgctgacgggaaacctgttcttcaggaatccgagtgggagtatacagacttttttgctgactccgaagacggtcaactcaacattgctgggatcacttatgtgaaggcctggattgtagagcgatcggacgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattcttaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagtgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagtttatcgccgaagtgccagagaagttggctgcggctgttacagtttttgtcaacttcttgaatgagctttttgagtctgcctgtgactgcttaaaggtcggaggtaaaacctttaacaaggttggctcctatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccttcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattatggcccatgaacaacaggatcttcaagggttcgtaaccacttgttgcacgatgtcaggttttgagtgttttatgcctataatcccacagtgtccagcagtgcttgaagagattgatggtggtagcatctggcgctcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggcggttgttttcacagtgttaaaagcgttctagcaagtgccttccagattcctgtccaggcaggcgttgagaagtttaaagtcttccttaactgtgttcaccctgttgtaccacgcgtcattgaaacttcttttgtggaattagaagagacgacatttaaaccaccagcactcaatggtagtattgctattgttgatggctttgctttctattatgatggaacactatactatcccaccgatggtaatagtgttgtgcctatctgttttaagaagaagggtggtggtgatgtcaaattctctgatgaagtctctgttagaaccattgacccagtttataaggtctcccttgaatttgagttcgagtctgagactattatggctgtgcttaataaggctgttggtaatcgtatcaaggttacaggtggttgggacgatgttgttgagtatatcaacgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgatggtcttaatgtagctcctgaaacaaatgtagagtctgaagttgaggaagttgccgcaaccttgtcctttattaaagatacaccttccacagttactaaagatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgagggttgtgcttttcgtatgacgccaactttggaaccgttcccatatggtgcttgtgctcagtgtgctcaagttttgatgcacacttttaaaagtattgttggcaccggcatcttttgtcgagatactactactctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcgcttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgaccggcggtgcattgcttgaagccataaatgtatttaaaaagagtaacaaaacagtgcctgctggcaactgtgttacttttgagtgtgcagacatgatttctattactattgtagtattgccatctgatggtgatgctaattatgacaaaaattatgcacgcgccgtcgtcaaggtatctaagcttaaaggcaagttattgcttgctgttggtgatgccacgttgtattccaagttgtcccacctcagcgtggtaggtttcgtatccacacctgatgatgtggagcgtttctacgcaaataagagtgtggttattaaagttactgaggatacacgtagtgttaaggctgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtccaagtacattgtttctgctggttctgtcactattgaacgtgtcacgcatgacggctgttgttgtagtaagcgtgttgtcactgcaccagttgtgaatgctagcgtattgaagcttggcgtcgaggatggtctttgtccacatggtcttaactacattgacaaagttgttgtagtcaaaggtactacaattgttgtcaatgttggaaaacctgtagtggcaccatcacacctctttcttaagggtgtttcttacacaacattcctagataatggtaacggtgttgtcggccattatactgtttttgatcatgacactggtatggtgcatgatggagatgcttttgtaccgggtgatctcaatgtatctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagcattttgggtctctgttttagggcctttcgtaagagggatgttaaagttctagctggtgtaccccaacgtactggtattatattgcgtaaaagtgtgcgctataatgcaaaggcgctgggtgtcttcttcaagctaaaactttattggttcaaagttcttggtaagtttagtttgggtatttatgcattgtatgcattattattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacattcttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagatagcaaatacacttgcaaagaatcacttaaaaattgtagtataatcacagactttattgtttttaacaataatggttccaatgtaaatcaggttaagaatgcatgtgtttatttttcacagatgctttgtaaacctgttaagttagtggactcagcgttgttggccagtttgtctgttgattttggtgcaagcttacatagtgcttttgttagtgtgttgtcgaatagttttggcaaagatctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttcggaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagctttcttgattttagtactcaggttagcagtgatagcgattatgacttcaagtatattgagagtggccagttgaagacttttgacaatccacttagttgtgtgcataatgtctttagtaacttcgaccagtggcatgaggccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgcaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttatcattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttctttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacctgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgctatgaatgtggcaaagagtgaatttgaccgtgaggcttctactcagcgtaagcttgatagaatggcggagcaggctgcagcacagatgtacaaagaggcaagagcagttaataggaagtccaaagttgtaagtgctatgcattcactgctttttggtatgttgagacgtttggatatgtcttctgtagacactattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagttaagtgggagttcgatggtggttgtaacactattgagctagaaccaccacgtaagttcttggtggattctcctaatggtgcacagatcaagtatctctactttgttcgtaaccttaacacgttacgtaggggtgccgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagtactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgcaatctatggtaacgtttgtagaaaggatcttaccgagtatactatgatggatttgtgttacgctttacgtaactttgatgaaaacaattgcgatgttcttaagagcattttaattaaggtaggtgcttgtgaggagtcctacttcaataataaagtctggtttgaccctgttgaaaatgaagacattcatcgtgtctatgcagtgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgctggatcgatggtgttccactggtaactacagctggttatcattttaaacagttaggtatagtttggaataatgaccttaacttacactctagcaggctctctattaacgaactactccagttttgtagtgatcctgcactgcttatagcatcatcaccagcccttgttgatcagcgtactgtttgcttttcagttgcagcgctaggtacaggtatgactaaccagactgtgaaacctggccatttcaataaggagttttatgacttcttacttgagcaaggtttcttctctgagggttctgagcttactttaaagcacttcttctttgcacagaagggtgatgcagctgttaaggattttgactactataggtataatagacccactgttctggacatttgccaagctcgcgtcgtgtatcaaatagtgcaacgctattttgatatttacgaaggtggttgtatcactgctaaagaagtggttgtcacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgctcttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggattacccaaagtgcgacagagcactgcccaatatgatacgcatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagttttcaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatacagattgtcgataaagatggtacttattaccttccttaccctgatccttcaagaatcctctctgcaggtgtgtttgttgatgacgttgttaaaactgatgcggttgtattgcttgaacgttatgtgtcattggctatagatgcctacccgttatctaagcatgaaaaccctgaatataagaaggtgttttatgtgcttttggattgggttaagcatctgtacaaaactttgaatgctggtgtgttagagtctttttctgtcacacttctggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaacacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctacgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcctccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtttacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttgggttttgacgttgaaggagcacatgttgttggctctaacgtcggtacaaatgtcccattgcaattagggttttctaacggtgttgattttgttgtcagacctgaaggttgcgttgtaactgagtctggtgactacattaaacccgtcagagctcgtgctccaccaggggaacaattcgcacaccttttgcctctacttaaacgcggccaaccatgggatgttgttcgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcacgtggcttctggtgatgccataatgactcgttgtctggccatacatgattgctttgtcaagaacgttgactggtccatcacatacccatttattggtaatgaggctgttattaataagagcggccgaattgtgcaatcacataccatgcggtcagttcttaagttatacaatccgaaagccatatatgatattggcaatcctaagggcatcagatgtgcagtaacggatgctaagtggttttgctttgacaagaatcctactaattctaatgtcaagacattggagtatgactatataacacatggccaatttgatgggttgtgcttgttttggaattgtaatgtggacatgtatccagaattttctgtggtctgtcgttttgacactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggagctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctctatgacccaaaatgctgtgcttatgtcacttacagccgttaaaaagcttactggcataaagttaacttatggttatcttaatggtgtcccagttaacacacatgaagataaaccttttacttggtatatttacactaggaagaacggcaaattcgaggactatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttacatctactaatttcgcaggtgcgtctgtcctgtatgggtgtgcttaaaatagacgagtttgtgtctagtaatgatagcacgttaaagtcctgtactgttacatatgctgataaccctagtagtaagatggtttgcacgtatatggatctcctgcttgacgattttgtcagcattcttaaatcgttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaagtttgagtattggacaatgttctgtaccagtgttaacacgtcatcgtcagaggcatttttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagcgcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacatctctgtcattcttgctgaaggctcaattgtacttcatactgctttaggaacaaatttttcttttgtttgcagtaattcctcagatcctcacttagccaccttcgccatacctctgggtgctatccaagtaccctattattgttttcttaaagtggatacttacaactccactgtttataaattcttggctgttttacctcctaccgtcagggaaattgtcatcaccaagtatggtgatgtttatgtcaatgggtttggctatttgcatctcggtttgttggacgctgtcacaattaatttcactggtcatggcactgacgatgacgtttcaggtttctggaccatagcatcgactaattttgttgatgcacttatcgaagttcaaggaactgccattcagcgtattctttattgtgatgatcctgttagccaactcaagtgttctcaggttgcttttgaccttgacgatggtttctaccctatttcttctagaaaccttctgagtcatgaacagccaatttcttttgttactttgccatcatttaatgatcattcttttgttaacattactgtctctgcgtcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagtaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccgagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggcgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccatcagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagctagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgggtgactttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaagctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccactggttgttgtggatgctgcggctgctgctgtgcttgtttttcaggttgttgtaggggtcctagacttcaacctttggcgctataaaaatgcgctctttattatttttaatactacgacactttctttcctcaatggtaaagcagcttattatgacggcaaatccattgtgattctagaaggtggtgaccattacatcacttttggcaactcttttgttgctttcgttagtagcattgacttgtatctagctatacgtgggcggcaagaagccgacctacagctgttgcgaactgttgagcttcttgatggcaagaagctttatgtcttttcgcaacatcaaattctcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccccagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagcctatcattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaacaaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggactttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgcctatttggaatgggacacagctgttgatggtggtgacacggccgttgaaattatcaacgagatcttcgacacaggaaattaaacaatgtttgactggcttaccctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgacttaaggactgttagtaactgaagacctgacggtgttgatatggatacac

SEQ ID NO: 11 - DNA, 26 bp, Artificial
  Primer
tcattactagtgttctgctgcatttc

SEQ ID NO: 12 - DNA, 26 bp, Artificial
  Primer
cacagattaaccaattggacgaaggt

(19) United States
(12) Patent Application Publication
(10) Pub. No.: US 2024/0285752 A1
(43) Pub. Date: Aug. 29, 2024

(54) Attenuated Porcine Epidemic Diarrhea Virus

(71) Applicant: Zoetis Services LLC, Parsippany, NJ (US)

(72) Inventors: Xiaosai RUAN, Changping District, Beijing (CN); Can LIU, Changping District, Beijing (CN); Haiyan LI, Suzhou (CN); Yanhong HOU, Beijing (CN); Hongxin YU, Qingdao (CN)

(21) Appl. No.: 18/248,573

(22) PCT Filed: Sep. 23, 2021
(86) PCT No.: PCT/US2021/051807

§ 371 Date: Apr. 11, 2023

(30) Foreign Application Priority Data
Sep. 29, 2020 (CN) 202011048314.0

(51) Int. Cl. A61K 39/215; A61K 39/00; A61P 31/14; C12N 7/00

(52) U.S. Cl. CPC A61K 39/215; A61P 31/14; C12N 7/00; A61K 2039/5254; A61K 2039/542; A61K 2039/552; C12N 2770/20021; C12N 2770/20034; C12N 2770/20062

(57) ABSTRACT

The disclosure provides a C-terminally truncated Spike protein of PEDV. Nucleic acid sequences including same and a virus comprising same, as well as methods of use are also provided.

### CROSS-REFERENCE TO RELATED APPLICATIONS

This application is a National Stage of PCT application PCT/US2021/051807 filed on Sep. 23, 2021, which derives priority from Chinese Application 202011048314.0 filed on Sep. 29, 2020.

### SEQUENCE LISTING INFORMATION

The instant application contains a Sequence Listing which has been submitted electronically in ASCII text file format and is hereby incorporated by reference in its entirety. Said ASCII text copy, created on Feb. 23, 2023, is named ZP000356A_ST25.txt and is 74,597 bytes in size.

### BACKGROUND

Porcine epidemic diarrhea (PED) is highly contagious and is characterized by dehydration, diarrhea, and high mortality in swine, particularly suckling piglets. The causative agent, porcine epidemic diarrhea virus (PEDV), is a single stranded, positive sense RNA virus belonging to the Alphacoronavirus genus of the family Coronaviridae. PEDV has a total genome size of approximately 28 kb and contains 7 open reading frames. Symptoms of PEDV infection are often similar to those caused by transmissible gastroenteritis virus (TGEV) and porcine deltacoronavirus (PDCoV), both of which are also members of the Coronaviridae, It should be noted that cross protection between PEDV and TGEV is not generally observed, the overall viral nucleotide sequences being at most about 60% similar.

PED was likely first observed in Europe circa 1970, and the causative virus was subsequently characterized (see for example M. Pensaert et al. Arch. Virol, v. 58, pp 243-247, 1978 and D. Chasey et al., Res. Vet Sci, v. 25, pp 255-256, 1978). PEDV was not identified in North America until 2013, at which point widespread outbreaks commenced, and severe economic losses to the swine industry resulted. The virus appeared in multiple, widely distributed sow herds within days, and it has spread to at least 32 states. Producers can expect losses of up to 100% in naive neonatal piglets. Present recommendations for management of infection include implementation of strict biosecurity and/or intentional exposure of the whole herd to PEDV to accomplish immunity.

PEDV caused widespread epidemics in several European countries during the 1970s and 1980s; but since the 1990s PED has become rare in Europe with occasional outbreak. This classical PEDV strain subsequently was spread to Asian countries such as Japan, China, South Korea, etc. Since 2010, severe PED epizootic outbreaks have been reported in China and the PEDV recovered from these outbreaks were genetically different from the classical PEDV strains. The initial PED outbreaks in U.S. swine had similar clinical presentations to those observed in China. Sequence analyses revealed that the original U.S. PEDVs (hereafter designated as U.S. PEDV prototype strain) are most genetically similar to some PEDVs circulating in China in 2011-2102. In January 2014, a PEDV variant strain, which has insertions and deletions (INDEL) in the spike gene compared to the U.S. PEDV prototype strains, was identified in the U.S. swine population. This variant strain was designated as U.S. PEDV S-INDEL-variant strain. After the PED outbreak in the U.S., detection of U.S. prototype-like PEDV has been reported in Canada, Mexico, Taiwan, South Korea, and Japan; detection of U.S. S-INDEL-variant-like PEDV has been reported in South Korea, Japan, Germany, Belgium, France, and Portugal. Currently, PEDV remains as a significant threat to the global swine industry. There remains a significant need for live, attenuated vaccines against PEDV, especially vaccines that can be effective upon oral administration.

### SUMMARY OF INVENTION

In one aspect, the invention provides a C-terminally truncated Spike protein of Porcine Epidemic Diarrhea Virus (PEDV), lacking SEQ ID NO: 1 (YEVFEKVHVQ) or a sequence comprising SEQ ID NO: 1 and comprising an amino acid sequence that is at least 90% identical to SEQ ID NO: 2 or a C-terminally truncated variant thereof, with a proviso that said C-terminally truncated Spike protein of PEDV is at least 1200 amino acids long.

According to different embodiments of this aspect, the C-terminally truncated Spike protein of PEDV may be at least 1250 amino acids long, or at least 1300 amino acids long, or at least 1370 amino acids long.

According to different embodiments of this aspect, the C-terminally truncated Spike protein of PEDV may be at least at least 95% identical to SEQ ID NO: 2, or at least 96%, or at least 97%, or at least 98%, or at least 99% identical, or 100% identical to SEQ ID NO: 2). In certain embodiments, the amino acids differing between SEQ ID NO: 2 and the sequence which is at least 90% identical thereto (i.e., at least 95%, at least 96%, at least 97%, at least 98% or at least 99%) are conservative substitutes.

In a second aspect, a nucleic acid sequence is disclosed, said nucleic acid sequence comprising a polynucleotide sequence encoding the C-truncated Spike protein of PEDV according any of the embodiments of the first aspect of the invention.

In a third aspect, the disclosure provides a virus that comprises the C-terminally truncated Spike protein of PEDV according to any embodiment of the first aspect of the invention, or the virus comprises the nucleic acid sequence according to any of the embodiments of the second aspect of the invention.

In a fourth aspect, the invention provides an amino acid sequence comprising SEQ ID NO: 5.

In a fifth aspect, the invention provides PEDV comprising the amino acid sequence according to the fourth aspect of the invention.

In a sixth aspect, the invention provides a C-terminally truncated Spike protein of PEDV, wherein said C-terminally truncated Spike protein is at least 90% identical to SEQ ID NO: 3 with the proviso that this C-terminally truncated Spike protein of PEDV comprises SEQ ID NO: 4. In different embodiments of this fifth aspect, the C-terminally truncated Spike protein of PEDV may be at least at least 95% identical to SEQ ID NO: 3, or at least 96% or at least 97% or at least 98% or at least 99% identical or 100% identical to SEQ ID NO: 3). In certain embodiments, the amino acids differing between SEQ ID NO: 2 and the sequence which is at least 90% identical thereto (i.e., at least 95%, at least 96%, at least 97%, at least 98% or at least 99%) are conservative substitutes.

In a seventh aspect, the invention provides a PEDV comprising ORF-2 and ORF 3, with a proviso that the virus comprises a first deletion in said ORF2/ORF3, wherein said first deletion is a deletion of SEQ ID NO: 6 or a deletion of a nucleic acid sequence comprising SEQ ID NO: 6, with a proviso that said virus expresses amino acid sequence comprising SEQ ID NO: 3 or a sequence that is at least 90% identical thereto, with a further proviso that C-terminal amino acids of said SEQ ID NO: 3 is QPLAL (SEQ ID NO: 4).

According to certain embodiments in this seventh aspect, the PEDV of the invention further comprises a second deletion in said ORF-3, wherein said second deletion is a deletion of SEQ ID NO: 7 or a deletion of a nucleic acid sequence comprising SEQ ID NO: 7. In certain embodiments, the first deletion and the second deletion are distinct.

In certain embodiments, the PEDV comprises wild-type ORFs encoding E, M, and N proteins. In certain embodiments, the PEDV of the invention lacks a functional protein expressed by ORF-3.

In certain embodiments, the virus has a genome according to SEQ ID NO: 10 or a sequence that is at least 90% identical thereto.

In some embodiments, the virus is derived from PEDV strain DJ.

In the eighth aspect, the invention provides a vaccine, wherein the vaccine comprises the virus according to any embodiments of the third, the fifth and/or the seventh aspect of the invention.

In certain embodiments of this eighth aspect, the virus is an attenuated virus.

In the ninth aspect, the invention provides a method of preventing a swine animal from PEDV infection comprising administering to said swine the vaccine according any embodiment of the eighth aspect of the invention.

In certain embodiments, the vaccine is administered orally.

In certain embodiments, the swine animal is a sow, wherein said vaccine is administered about 28-42 days before the farrowing and wherein further said vaccine is administered about 17-21 days before the farrowing. In certain embodiments, the first and/or the second vaccinations administered orally.

In the tenth aspect, the invention provides method of protecting a piglet from PEDV infection comprising administering to said piglet colostrum from a sow vaccinated with the vaccine according any embodiment of the eighth aspect of the invention, wherein the sow is vaccinated about 28-42 days before the farrowing (e.g., 35 days) and wherein further said vaccine is administered about 7-21 (e.g., 14 days) days before the farrowing. In certain embodiments, the first and/or the second vaccinations administered orally.

In certain embodiments, said piglet is at least 3 days old. In other embodiments, the piglet is at least five days old.

### BRIEF DESCRIPTION OF THE DRAWINGS

FIG. 1 is an electrophoretic map of nucleic acid of continuously passaged virus.

FIG. 2 is an electrophoretic map of nucleic acid of 5 continuously passaged virus.

FIG. 3 is an illustration of anti PEDV antibody levels in vaccinated sows at the time of farrowing.

FIG. 4 is an illustration of anti PEDV antibody levels in 3-5-day-old piglets fed with colostrum from vaccinated sows.

### DETAILED DESCRIPTION

For the better understanding of the invention, the following definitions are provided:

The term “about” as applied to a reference number refers to the reference number plus or minus 10 of said value.

The term “adjuvant” refers to a compound that enhances the effectiveness of the vaccine, and may be added to the formulation that includes the immunizing agent. Adjuvants provide enhanced immune response even after administration of only a single dose of the vaccine. Adjuvants may include, for example, muramyl dipeptides, pyridine, aluminum hydroxide, dimethyldioctadecyl ammonium bromide (DDA), oils, oil-in-water emulsions, saponins, cytokines, and other substances known in the art. Examples of suitable adjuvants are described in U.S. Patent Application Publication No. US2004/0213817 A1. “Adjuvanted” refers to a composition that incorporates or is combined with an adjuvant.

An “attenuated” PEDV as used herein refers to a PEDV which is capable of infecting and/or replicating in a susceptible host, but is non-pathogenic or less-pathogenic to the susceptible host. For example, the attenuated virus may cause no observable/detectable clinical manifestations, or less clinical manifestations, or less severe clinical manifestations, or exhibit a reduction in virus replication efficiency and/or infectivity, as compared with the related field isolated strains. The clinical manifestations of PEDV infection can include, without limitation, clinical diarrhea, vomiting, lethargy, loss of condition and dehydration.

The term “conservative substitutions” refers to replacement of one amino acid with another amino acid with similar properties. The skilled person will further acknowledge that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K), Histidine (H); [5] Isoleucine (I), Leucine (L), Methionine (M), Valine (V); and [6] Phenylalanine (F), Tyrosine (Y), Tryptophan (W), (see, e.g., US Patent Publication 20100291549).

An “epitope” is an antigenic determinant that is immunologically active in the sense that once administered to the host, it is able to evoke an immune response of the humoral (B cells) and/or cellular type (T cells). These are particular chemical groups or peptide sequences on a molecule that are antigenic. An antibody specifically binds a particular antigenic epitope on a polypeptide. In the animal most antigens will present several or even many antigenic determinants simultaneously. Such a polypeptide may also be qualified as an immunogenic polypeptide and the epitope may be identified as described further.

The term “immunogenic fragment” as used herein refers to a polypeptide or a fragment of a polypeptide, or a nucleotide sequence encoding the same which comprises an allele-specific motif, an epitope or other sequence such that the polypeptide or the fragment will bind an MHC molecule and induce a cytotoxic T lymphocyte (“CTL”) response, and/or a B cell response (for example, antibody production), and/or T-helper lymphocyte response, and/or a delayed type hypersensitivity (DTH) response against the antigen from which the immunogenic polypeptide or the immunogenic fragment is derived. A DTH response is an immune reaction in which T cell-dependent macrophage activation and inflammation cause tissue injury. A DTH reaction to the subcutaneous injection of antigen is often used as an assay for cell-mediated immunity.

With the term “induction of an immunoprotective response” is meant a (humoral and/or cellular) immune response that reduces or eliminates one or more of the symptoms of disease, i.e. clinical signs, lesions, bacterial excretion and bacterial replication in tissues in the infected subject compared to a healthy control. Preferably said reduction in symptoms is statistically significant when compared to a control.

A “pharmaceutically acceptable carrier” means any conventional pharmaceutically acceptable carrier, vehicle, or excipient that is used in the art for production and administration of vaccines. Pharmaceutically acceptable carriers are typically non-toxic, inert, solid or liquid carriers.

The terms “porcine” and “swine” are used interchangeably herein and refer to any animal that is a member of the family Suidae such as, for example, a pig.

A “susceptible” host as used herein refers to a cell or an animal that can be infected by PEDV. When introduced to a susceptible animal, an attenuated PEDV may also induce an immunological response against the PEDV or its antigen, and thereby render the animal immunity against PEDV infection.

“Therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

“Treating” refers to preventing a disorder, condition, or disease to which such term applies, or to preventing or reducing one or more symptoms of such disorder, condition, or disease.

The term “vaccine” refers to an antigenic preparation used to produce immunity to a disease, in order to prevent or ameliorate the effects of infection. Vaccines are typically prepared using a combination of an immunologically effective amount of an immunogen together with an adjuvant effective for enhancing the immune response of the vaccinated subject against the immunogen.

PEDV is an enveloped virus possessing approximately a 28 kb, positive-sense, single stranded RNA genome, with a 5′ cap and a 3′ polyadenylated tail. (Pensaert and De Bouck P. 1978). The genome comprises a 5′ untranslated region (UTR), a 3′ UTR, and at least seven open reading frames (ORFs) that encode four structural proteins (spike (S), envelope (E), membrane (M), and nucleocapsid (N)) and three non-structural proteins (replicases 1a and 1b and ORF3); these are arranged on the genome in the order 5′-replicase (1a/1b)-ORF2 (also known as S)-ORF3-E-M-N-3′ (Oldham J. 1972; and Bridgen et al. 1993). The first three emergent North American PEDV genomic sequences characterized, Minnesota MN (GenBank: KF468752.1), Iowa IA1 (GenBank: KF468753.1), and Iowa IA2 (GenBank: KF468754.1), have the same size of 28,038 nucleotides (nt), excluding the polyadenosine tail and share the genome organization with the prototype PEDV CV777 strain (GenBank: AF353511.1). These three North American PEDV sequences shared 99.8 to 99.9% nucleotide identities. In particular, strains MN and IA2 had only 11 nucleotide differences across the entire genome.

For the purposes of the application, the sequences are provided in DNA format. A person of ordinary skill in the art would have no difficulties in translating these sequences into RNA sequences which comprise the genome of the virus.

The inventors have surprisingly discovered that a PEDV having a first deletion in a region of ORF-2/ORF-3 results in a virus that is attenuated and immunogenic—i.e., generates protective response against wild-type PED. In certain embodiments, the first deletion comprises SEQ ID NO: 6. This sequence starts in ORF-2 and spans a proximal portion of ORF3 including the ORF-3 start codon. The first deletion is not limited to SEQ ID NO: 6 only and can include sequences upstream or downstream of SEQ ID NO: 6. It is noted however, that since Spike protein encoded by ORF2 is a major immunogen of PED, the first deletion may not extend so far upstream of SEQ ID NO: 6 as to compromise the spike protein.

Thus, the invention provides a fragment of a Spike protein. The fragment of the Spike protein lacks SEQ ID NO: 1. The fragment may be further C-terminally truncated but it should be generally at least 1200 amino acids long, preferably at least 1300 amino acids long, and more preferably, at least 1370 amino acids long. In certain embodiments, the fragment of the Spike protein comprises SEQ ID NO: 2, or sequences that are at least 90% (or at least 95%, 96%, 97%, 98%) identical thereto. It is preferred that the amino acids differing between the sequence that is at least 90% identical to SEQ ID NO: 2 and SEQ ID NO: 2 itself are conservative substitutions.

One of ordinary skill in the art can appreciate that the first deletion causes a frameshift in ORF-2 and thus alters the C-terminal amino acid sequence of the wild-type Spike protein. The Spike protein fragment according to the invention lacks SEQ ID NO: 1. Instead, in the most preferred embodiment, the spike protein fragment ends with QPLAL (SEQ ID NO: 4).

In the most preferred set of embodiments, the spike protein fragment described herein comprises SEQ ID NO: 3, or a sequence that is at least 90% identical thereto, with a proviso that SEQ ID NO: 4 is present at the C-terminus of said spike protein fragment or the sequence that is at least 90% identical to SEQ ID NO: 3. The sequence identity may be greater (for example, at least 95%, 96%, 97%, 98%, or 99%) and the differing amino acid are conservative substitutions.

Techniques to obtain the polypeptides according to the invention are well known in the art. For example, genetic engineering techniques and recombinant DNA expression systems may be used.

In another aspect, the invention provides a nucleic acid sequence encoding the Spike protein fragment according to any of the embodiments described above. Nucleic acid molecules encoding the amino acid sequences according to any embodiment of the first aspect of the invention may also be inserted into a vector (e.g., a recombinant vector) such as one or more non-viral and/or viral vectors. Non-viral vectors may include, for instance, plasmid vectors (e.g., compatible with bacterial, insect, and/or mammalian host cells). Exemplary vectors may include, for example, PCR-ii, PCR3, and pcDNA3.1 (Invitrogen, San Diego, Calif.), pBSii (Stratagene, La Jolla, Calif.), pet15 (Novagen, Madison, Wis.), pGEX (Pharmacia Biotech, Piscataway, N.J.), pEGFp-n2 (Clontech, Palo Alto, Calif.), pET1 (Bluebacii, Invitrogen), pDSR-alpha (PCT pub. No. WO 90/14363) and pFASTBACdual (Gibco-BRL, Grand island, NY) as well as Bluescript plasmid derivatives (a high copy number COLe1-based phagemid, Stratagene Cloning Systems, La Jolla, Calif.), PCR cloning plasmids designed for cloning TAQ-amplified PCR products (e.g., TOPO™ TA Cloning® kit, PCR2.1® plasmid derivatives, Invitrogen, Carlsbad, Calif.). Bacterial vectors may also be used including, for instance, Shigella, Vibrio cholerae, Lactobacillus, Bacille Calmette Guerin (BCG), and Streptococcus (see for example, WO 88/6626; WO 90/0594; WO 91/13157; WO 92/1796; and WO 92/21376). The vectors may be constructed using standard recombinant techniques widely available to one skilled in the art. Many other non-viral plasmid expression vectors and systems are known in the art and may be used.

In the third aspect, the invention provides a vector comprising the nucleic acid sequence according to the second aspect of the invention. Various viral vectors that have been successfully utilized for introducing a nucleic acid to a host include retrovirus, adenovirus, adeno-associated virus (AAV), herpes virus, and poxvirus, among others. Viral vectors may be constructed using standard recombinant techniques widely available to one skilled in the art. See, e.g., Molecular cloning: a laboratory manual (Sambrook & Russell: 2000, Cold Spring Harbor Laboratory Press; ISBN: 0879695773), and: Current protocols in molecular biology (Ausubel et al., 1988+ updates, Greene Publishing Assoc., New York; ISBN: 0471625949).

In certain embodiments, the vector is a viral vector, and the virus is PEDV.

Thus, the invention provides a PEDV which comprises the first deletion and/or the spike protein fragment described above. One may appreciate that the first deletion includes the start codon on ORF-3 thereby eliminating said ORF. The PEDV of the invention thus lacks a functional protein expressed by a wild-type ORF-3.

However, due to the deletion, a new ORF is created, referred to as “new ORF” or “newly created ORF” or the like. Conceptual translation of this new ORF (SEQ ID NO: 9) by ORF Finder software publicly available from NCBI website reveals that SEQ ID NO: 5 is the product of expression of this new ORF. Thus, in another aspect, the invention provides a PEDV which expresses the amino acid sequence of SEQ ID NO: 5 and, in certain embodiments, comprises an ORF of SEQ ID NO: 9.

The methods of protein and/or nucleic acid sequence identities described above are applicable to all proteins and/or nucleic acids of the described herein. Multiple sequence comparison algorithms and programs for evaluating sequence identities and/or similarities are known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by comparing sequence information using the computer program GAP, i.e., Genetics Computer Group (GCG; Madison, WI) Wisconsin package version 10.0 program, GAP (Devereux et al. (1984), Nucleic Acids Res. 12: 387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

In certain embodiments, the virus according to the invention, also comprises a second deletion in the sequence that is a part of the wild-type ORF 3. Preferably, this second deletion comprises (or consists of) SEQ ID NO: 7.

In the certain preferred embodiments, PEDV is provided, wherein said virus comprises a first deletion consisting of SEQ ID NO: 6 and a second deletion consisting of SEQ ID NO: 7. In a more preferable set of embodiments, the genomic sequence of the virus comprises SEQ ID NO: 10 or a sequence that is 90% identical thereto (e.g., 95%, 96%, 97%, 98%, 99%, 99.5% or greater). Preferably, the differing nucleotides do not result in significant (or any) changes in the expressed amino acid sequences and are results of codon optimization. The amino acid sequences of E, M, and N proteins of the PEDV of the invention are, preferably, not altered compared to the wild-type virus, which may belong to Genotype 1 or Genotype 2. A non-limiting example of PEDV Genotype 1 is CV777 (GenBank Accession no. AF353511), and non-limiting examples of Genotype 2 are DJ strain as well as AJ1102 strain (GenBank Accession no. JX188454). Additional non-limiting examples of Genotype 2 strains include strains CH/ZJCS03/2012, CH/JXZS03/2014, CH/JXFX01/2014, CH/JXJJ08/2015, CH/JXGZ04/2015, CH/JXJA89/2015, CH/JXDX119/2016, CH/JXJGS11/2016, CH/JXWN13/2016, CH/JXJJ18/2017, CH/JXNC38/2017, CH/JX/01, CH/JX-1/2013, CH/JX-2/2013, AH2012, GD-B, BJ-2011-1, CH/FJND-3/2011, AJ1102, GD-A, CH/GDGZ/2012, CH/ZJCX-1/2012, CH/FJZZ-9/2012. Genotype 2 is the dominant genotype in the field from 2010 to 2020 in China and neighboring countries. The virus according to the invention may be derived from these and other parental strains by culture passaging or by introducing the mutations described above by genetic engineering techniques. In other embodiments, the virus described above may be further attenuated by cell culture passaging. Thus, in other embodiments, said further attenuated virus is a progeny of the virus having genomic sequence that comprises SEQ ID NO: 10 or a sequence that is 90% identical thereto.

The present invention preferably includes vaccine compositions comprising a live, attenuated variant PEDV of the invention and a pharmaceutically acceptable carrier. As used herein, the expression “live, attenuated PEDV of the invention” encompasses any live, attenuated PEDV strain that includes one or more of the variations described herein. The pharmaceutically acceptable carrier can be, e.g., water, a stabilizer, a preservative, culture medium, or a buffer. Vaccine formulations comprising the attenuated PEDV of the invention can be prepared in the form of a suspension or in a lyophilized form or, alternatively, in a frozen form. If frozen, glycerol or other similar agents may be added to enhance stability when frozen. The advantages of live attenuated vaccines, in general, include the presentation of all the relevant immunogenic determinants of an infectious agent in its natural form to the host's immune system, and the need for relatively small amounts of the immunizing agent due to the ability of the agent to multiply in the vaccinated host.

Attenuation of the virus for a live vaccine, so that it is insufficiently pathogenic to substantially harm the vaccinated target animal, may be accomplished by known procedures, including preferably by serial passaging. The following references provide various general methods for attenuation of coronaviruses, and are suitable for attenuation or further attenuation of any of the strains useful in the practice of the present invention: B. Neuman et al., Journal of Virology, vol. 79, No. 15, pp. 9665-9676, 2005; J. Netland et al., Virology, v 399(1), pp. 120-128, 2010; Y-P Huang et al., “Sequence changes of infectious bronchitis virus isolates in the 3′ 7.3 kb of the genome after attenuating passage in embryonated eggs, Avian Pathology, v. 36 (1), (Abstract), 2007; and S. Hingley et al., Virology, v. 200(1) 1994, pp. 1-10; see U.S. Pat. No. 3,914,408; and Ortego et al., Virology, vol. 308 (1), pp. 13-22, 2003.

Additional genetically engineered vaccines, which are desirable in the present invention, are produced by techniques known in the art. Such techniques involve, but are not limited to, further manipulation of recombinant DNA, modification of or substitutions to the amino acid sequences of the recombinant proteins and the like.

Genetically engineered vaccines based on recombinant DNA technology are made, for instance, by identifying alternative portions of the viral gene encoding proteins responsible for inducing a stronger immune or protective response in pigs (e.g., proteins derived from M, GP2, GP3, GP4, or GP5, etc.). Various subtypes or isolates of the viral protein genes can be subjected to the DNA-shuffling method. The resulting heterogeneous chimeric viral proteins can be used broad protecting subunit vaccines. Alternatively, such chimeric viral genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for any residual virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

An immunologically effective amount of the vaccines of the present invention is administered to a pig in need of protection against viral infection. The immunologically effective amount or the immunogenic amount that inoculates the pig can be easily determined or readily titrated by routine testing. An effective amount is one in which a sufficient immunological response to the vaccine is attained to protect the pig exposed to the PEDV. Preferably, the pig is protected to an extent in which one to all of the adverse physiological symptoms or effects of the viral disease are significantly reduced, ameliorated or totally prevented.

Vaccines of the present invention can be formulated following accepted convention to include acceptable carriers for animals, such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

The vaccines according to the invention may be administered in a variety of way, including without limitations, orally, subcutaneously, intramuscularly, itradermally, intravenously and the like.

The vaccines of the invention formulated for the mucosal administration (orally, intranasally, rectally) may be formulated with a mucoadhesive agent such as chitosan.

Vaccines of the present invention formulated for administration by injection or infusion may further comprise one or more additional immunomodulatory components such as, e.g., an adjuvant or cytokine, among others. Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the invention include modified SEAM®62 and SEAM® 1/2 formulations. Modified SEAM®62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) TWEEN®80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines.

Additional adjuvant systems permit for the combination of both T-helper and B-cell epitopes, resulting in one or more types of covalent T-B epitope linked structures, with may be additionally lipidated, such as those described in WO2006/084319, WO2004/014957, and WO2004/014956.

In certain embodiments of the present invention, ORFI PEDV protein, or other PEDV proteins or fragments thereof, is formulated with 5% AMPHIGEN® as discussed hereinafter.

A preferred adjuvanted may be provided as a 2 ML dose in a buffered solution further comprising about 5% (v/v) REHYDRAGEL® (aluminum hydroxide gel) and “20% AMPHIGEN” ® at about 25% final (v/v). AMPHIGEN® is generally described in U.S. Pat. No. 5,084,269 and provides de-oiled lecithin (preferably soy) dissolved in a light oil, which is then dispersed into an aqueous solution or suspension of the antigen as an oil-in-water emulsion. AMPHIGEN has been improved according to the protocols of U.S. Pat. No. 6,814,971 (see columns 8-9 thereof) to provide a so-called “20% AMPHIGEN” component for use in the final adjuvanted vaccine compositions of the present invention. Thus, a stock mixture of 10% lecithin and 90% carrier oil (DRAKEOL®, Penreco, Karns City, Pa.) is diluted 1:4 with 0.63% phosphate buffered saline solution, thereby reducing the lecithin and DRAKEOL® components to 2% and 18% respectively (i.e. 20% of their original concentrations). TWEEN®80 and SPAN®80 surfactants are added to the composition, with representative and preferable final amounts being 5.6% (v/v) TWEEN®80 and 2.4% (v/v) SPAN®80, wherein the Span is originally provided in the stock DRAKEOL® component, and the SPAN® is originally provided from the buffered saline component, so that mixture of the saline and DRAKEOL® components results in the finally desired surfactant concentrations. Mixture of the DRAKEOL®/lecithin and saline solutions can be accomplished using an In-Line Slim Emulsifier apparatus, model 405, Charles Ross and Son, Hauppauge, N.Y., USA.

The vaccine composition may also include REHYDRAGEL® LV (about 2% aluminum hydroxide content in the stock material), as additional adjuvant component (available from Reheis, N.J., USA, and ChemTrade Logistics, USA). With further dilution using 0.63% PBS, the final vaccine composition contains the following compositional amounts per 2 ML dose; 5% (v/v) REHYDRAGEL® LV; 25% (v/v) of “20% AMPHIGEN”, i.e. it is further 4-fold diluted); and 0.01% (w/v) of merthiolate.

As is understood in the art, the order of addition of components can be varied to provide the equivalent final vaccine composition. For example, an appropriate dilution of virus in buffer can be prepared. An appropriate amount of REHYDRAGEL® LV (about 2% aluminum hydroxide content) stock solution can then be added, with blending, in order to permit the desired 5% (v/v) concentration of REHYDRAGEL® LV in the actual final product. Once prepared, this intermediate stock material is combined with an appropriate amount of “20% AMPHIGEN” stock (as generally described above, and already containing necessary amounts of TWEEN® 80 and SPAN® 80) to again achieve a final product having 25% (v/v) of “20% AMPHIGEN”. An appropriate amount of 10% merthiolate can finally be added.

The vaccinate compositions of the invention permit variation in all of the ingredients, such that the total dose of antigen may be varied preferably by a factor of 100 (up or down) compared to the antigen dose stated above, and most preferably by a factor of 10 or less (up or down). Similarly, surfactant concentrations (whether Tween or Span) may be varied by up to a factor of 10, independently of each other, or they may be deleted entirely, with replacement by appropriate concentrations of similar materials, as is well understood in the art.

REHYDRAGEL® concentrations in the final product may be varied, first by the use of equivalent materials available from many other manufacturers (i.e. ALHYDROGEL®, Brenntag; Denmark), or by use of additional variations in the REHYDRAGEL®. line of products such as CG, HPA or HS. Using LV as an example, final useful concentrations thereof including from 0% to 20%, with 2-12% being more preferred, and 4-8% being most preferred, Similarly, the although the final concentration of AMPHIGEN® (expressed as % of “20% AMPHIGEN”) is preferably 25%, this amount may vary from 5-50%, preferably 20-30% and is most preferably about 24-26%.

The immunogenic and vaccine compositions of the invention can further comprise pharmaceutically acceptable carriers, excipients and/or stabilizers (see e.g. Remington: The Science and practice of Pharmacy, 2005, Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury((o-carboxyphenyl)thio)ethyl sodium salt (THIOMERSAL®), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN® or PLURONICS®.

Vaccines of the present invention, whether formulated for the administration by injection or for the administration to mucous surfaces (orally, intranasally, rectally and the like), can optionally be formulated for sustained release of the virus, infectious DNA molecule, plasmid, or viral vector of the present invention. Examples of such sustained release formulations include virus, infectious DNA molecule, plasmid, or viral vector in combination with composites of biocompatible polymers, such as, e.g., poly (lactic acid), poly (lactic-co-glycolic acid), methylcellulose, hyaluronic acid, collagen and the like. The structure, selection and use of degradable polymers in drug delivery vehicles have been reviewed in several publications, including A. Domb et al., 1992, Polymers for Advanced Technologies 3: 279-292, which is incorporated herein by reference. Additional guidance in selecting and using polymers in pharmaceutical formulations can be found in texts known in the art, for example M. Chasin and R. Langer (eds), 1990, “Biodegradable Polymers as Drug Delivery Systems” in: Drugs and the Pharmaceutical Sciences, Vol. 45, M. Dekker, NY, which is also incorporated herein by reference. Alternatively, or additionally, the virus, plasmid, or viral vector can be microencapsulated to improve administration and efficacy. Methods for microencapsulating antigens are well-known in the art, and include techniques described, e.g., in U.S. Pat. Nos. 3,137,631; 3,959,457; 4,205,060; 4,606,940; 4,744,933; 5,132,117; and International Patent Publication WO 95/28227, all of which are incorporated herein by reference.

Liposomes can also be used to provide for the sustained release of virus, plasmid, viral protein, or viral vector. Details concerning how to make and use liposomal formulations can be found in, among other places, U.S. Pat. Nos. 4,016,100; 4,452,747; 4,921,706; 4,927,637; 4,944,948; 5,008,050; and 5,009,956, all of which are incorporated herein by reference.

An effective amount of any of the above-described vaccines can be determined by conventional means, starting with a low dose of virus, viral protein plasmid or viral vector, and then increasing the dosage while monitoring the effects. An effective amount may be obtained after a single administration of a vaccine or after multiple administrations of a vaccine. Known factors can be taken into consideration when determining an optimal dose per animal. These include the species, size, age and general condition of the animal, the presence of other drugs in the animal, and the like. The actual dosage is preferably chosen after consideration of the results from other animal studies.

One method of detecting whether an adequate immune response has been achieved is to determine seroconversion and antibody titer in the animal after vaccination. The timing of vaccination and the number of boosters, if any, will preferably be determined by a doctor or veterinarian based on analysis of all relevant factors, some of which are described above.

The effective dose amount of virus, protein, infectious nucleotide molecule, plasmid, or viral vector, of the present invention can be determined using known techniques, taking into account factors that can be determined by one of ordinary skill in the art such as the weight of the animal to be vaccinated. The dose amount of virus of the present invention in a vaccine of the present invention preferably ranges from about 10¹ to about 10⁹ pfu (plaque forming units), more preferably from about 10² to about 10⁸ pfu, and most preferably from about 10³ to about 10⁷ pfu. The dose amount of a plasmid of the present invention in a vaccine of the present invention preferably ranges from about 0.1 μg to about 100 mg, more preferably from about 1 μg to about 10 mg, even more preferably from about 10 μg to about 1 mg. The dose amount of an infectious DNA molecule of the present invention in a vaccine of the present invention preferably ranges from about 0.1 μg to about 100 mg, more preferably from about 1 μg to about 10 mg, even more preferably from about 10 μg to about 1 mg. The dose amount of a viral vector of the present invention in a vaccine of the present invention preferably ranges from about 10¹ pfu to about 10⁹ pfu, more preferably from about 10² pfu to about 10⁸ pfu, and even more preferably from about 10³ to about 10⁷ pfu. A suitable dosage size ranges from about 0.5 ml to about 10 ml, and more preferably from about 1 ml to about 5 ml.

Suitable doses for viral protein or peptide vaccines according to the practice of the present invention (e.g., of the Spike protein fragment as discussed above) range generally from 1 to 50 micrograms per dose, or higher amounts as may be determined by standard methods, with the amount of adjuvant to be determined by recognized methods in regard of each such substance. In a preferred example of the invention relating to vaccination of swine, an optimum age target for the animals is between about 1 and 21 days, which at pre-weening, may also correspond with other scheduled vaccinations such as against Mycoplasma hyopneumoniae. Additionally, a preferred schedule of vaccination for breeding sows would include similar doses, with an annual revaccination schedule.

### Dosing

A preferred clinical indication is for treatment, control and prevention in both breeding sows and gilts pre-farrowing, followed by vaccination of piglets. In a representative example (applicable to both sows and gilts), two 2-ML doses of vaccine will be used, although of course, actual volume of the dose is a function of how the vaccine is formulated, with actual dosing amounts ranging from 0.1 to 5 ML, taking also into account the size of the animals. Single dose vaccination is also appropriate.

The first dose may be administered as early as pre-breeding to 5-weeks pre-farrowing, with the second dose administered preferably at about 1-3 weeks pre-farrowing. Doses vaccine preferably provide an amount of viral material that corresponds to a TCID₅₀ (tissue culture infective dose) of between about 10⁶ and 10⁸, more preferably between about 10⁷ and 10^{7.5}, and can be further varied, as is recognized in the art. Booster doses can be given two to four weeks prior to any subsequent farrowings. Intramuscular vaccination (all doses) is preferred, although one or more of the doses could be given subcutaneously. Oral administration is also preferred. Vaccination may also be effective in naive animals, and non-naive animals as accomplished by planned or natural infections.

In a further preferred example, the sow or gilt is vaccinated intramuscularly or orally at about 8-weeks pre-farrowing and then 2-weeks pre-farrowing. Under these conditions, a protective immune response can be demonstrated in PEDV-negative vaccinated sows in that they developed antibodies (measured via fluorescent focal neutralization titer from serum samples) with neutralizing activity, and these antibodies were passively transferred to their piglets. The protocols of the invention are also applicable to the treatment of already seropositive sows and gilts, and also piglets and boars. Booster vaccinations can also be given and these may be via the same or a different route of administration. Although it is preferred to re-vaccinate a mother sow prior to any subsequent farrowings, the vaccine compositions of the invention nonetheless can still provide protection to piglets via ongoing passive transfer of antibodies, even if the mother sow was only vaccinated in association with a previous farrowing.

It should be noted that piglets may then be vaccinated as early as Day 1 of life. For example, piglets can be vaccinated at Day 1, with or without a booster dose at 3 weeks of age, particularly if the parent sow, although vaccinated pre-breeding, was not vaccinated pre-farrowing. Piglet vaccination may also be effective if the parent sow was previously not naive either due to natural or planned infection. Vaccination of piglets when the mother has neither been previously exposed to the virus, nor vaccinated pre-farrowing may also effective. Boars (typically kept for breeding purposes) should be vaccinated once every 6 months. Variation of the dose amounts is well within the practice of the art. It should be noted that the vaccines of the present invention are safe for use in pregnant animals (all trimesters) and neonatal swine. The vaccines of the invention are attenuated to a level of safety (i.e. no mortality, only transient mild clinical signs or signs normal to neonatal swine) that is acceptable for even the most sensitive animals again including neonatal pigs. Of course, from a standpoint of protecting swine herds both from PEDV epidemics and persistent low level PEDV occurrence, programs of sustained sow vaccination are of great importance. It will be appreciated that sows or gilts immunized with PEDV MLV will passively transfer immunity to piglets, including PEDV-specific IgA, which will protect piglets from PEDV associated disease and mortality. Additionally, generally, pigs that are immunized with PEDV MLV will have a decrease in amount and/or duration or be protected from shedding PEDV in their feces, and further, pigs that are immunized with PEDV MLV will be protected from weight loss and failure to gain weight due to PEDV, and further, PEDV MLV will aid in stopping or controlling the PEDV transmission cycle.

It should also be noted that animals vaccinated with the vaccines of the invention are also immediately safe for human consumption, without any significant slaughter withhold, such as 21 days or less.

When provided therapeutically, the vaccine is provided in an effective amount upon the detection of a sign of actual infection. Suitable dose amounts for treatment of an existing infection include between about 10⁶ and about 10⁹ TCID₅₀, or higher, of virus per dose (minimum immunizing dose to vaccine release). A composition is said to be “pharmacologically acceptable” if its administration can be tolerated by a recipient. Such a composition is said to be administered in a “therapeutically or prophylactically effective amount” if the amount administered is physiologically significant.

At least one vaccine or immunogenic composition of the present invention can be administered by any means that achieve the intended purpose, using a pharmaceutical composition as described herein. For example, route of administration of such a composition can be by parenteral, oral, oronasal, intranasal, intratracheal, topical, subcutaneous, intramuscular, transcutaneous, intradermal, intraperitoneal, intraocular, and intravenous administration. In one embodiment of the present invention, the composition is administered by intramuscularly. Parenteral administration can be by bolus injection or by gradual perfusion over time. Any suitable device may be used to administer the compositions, including syringes, droppers, needleless injection devices, patches, and the like. The route and device selected for use will depend on the composition of the adjuvant, the antigen, and the subject, and such are well known to the skilled artisan. Administration that is oral, or alternatively, subcutaneous, is preferred. Oral administration may be direct, via water, or via feed (solid or liquid feed). When provided in liquid form, the vaccine may be lyophilized with reconstitution, or provided as a paste, for direct addition to feed (mix in or top dress) or otherwise added to water or liquid feed.

In yet another aspect, the proteins, the nucleic acid sequences, and the viruses of the invention would allow a person of ordinary skill in the art to differentiate the previously infected animals and the animals vaccinated with the vaccines described above. For example, antibodies can be made that bind the truncated S protein fragment according to the invention (e.g., by targeting SEQ ID NO: 4) but not the wild-type S-protein. Antibodies can also be made against the amino acid sequence expressed by the new ORF (SEQ ID NO: 5). The methods of making antibodies are well known in the art and one of ordinary skill in the art would not have to engage in undue experimentation to prepare the polyclonal or monoclonal antibodies suitable for the invention. The isolated proteins according to the invention can also be prepared. For example, reaction of SEQ ID NO: 5 with an antibody from a blood sample from the tested animals would suggest that the animal was vaccinated.

In other embodiments, the presence of the virus according to the invention in a sample from the tested animal can be detected using the primers that target the first or the second deletion. For example, if the primers are designed to amplify the region within the first deletion, absence of the PCR reaction product may indicate the presence of the virus according to the invention in the sample.

The disclosure also provides the following items:

Item 1. A C-terminally truncated Spike protein of Porcine Epidemic Diarrhea (PED) virus lacking SEQ ID NO: 1 (YEVFEKVHVQ) or a sequence comprising SEQ ID NO: 1 and comprising an amino acid sequence that is at least 90% identical to SEQ ID NO: 2 or a C-terminally truncated variant thereof, with a proviso that said C-terminally truncated Spike protein of PEDV is at least 1200 amino acids long.

Item 2. The C-terminally truncated Spike protein of PEDV according to item 1, with a proviso that said C-terminally truncated Spike protein of PEDV is at least 1250 amino acids long.

Item 3. The C-terminally truncated Spike protein of PEDV according to item 1, with a proviso that said C-terminally truncated Spike protein of PEDV is at least 1300 amino acids long.

Item 4. The C-terminally truncated Spike protein of PEDV according to item 1, with a proviso that said C-terminally truncated Spike protein of PEDV is at least 1370 amino acids long.

Item 5. The C-terminally truncated Spike protein according to any one of items 1-4 comprising an amino acid sequence that is at least 95% identical to SEQ ID NO: 2.

Item 6. The C-terminally truncated Spike protein according to any one of items 1-4 comprising an amino acid sequence that is at least 99% identical to SEQ ID NO: 2.

Item 7. The C-terminally truncated Spike protein according to any one of items 4-6, which is a conservatively substituted variant of SEQ ID NO: 2.

Item 8. A nucleic acid sequence encoding the C-terminally truncated Spike protein according to any one of items 1-7.

Item 9. A virus comprising the C-terminally truncated Spike protein of any one of claims 1-7 or the nucleic acid sequence of item 8.

Item 10. An amino acid sequence comprising SEQ ID NO: 3 or a sequence that is at least 90% identical thereto, with a proviso that C-terminal amino acids of said SEQ ID NO: 3 is QPLAL (SEQ ID NO: 4).

Item 11. The amino acid sequence of item 10, wherein the sequence is at least 95% identical to SEQ ID NO: 3.

Item 12. The amino acid sequence of item 10, wherein the sequence is at least 99% identical to SEQ ID NO: 3.

Item 13. The amino acid sequence of any one of items 10-12 which is a conservatively substituted variant of SEQ ID NO: 3.

Item 14. A nucleic acid sequence encoding the amino acid sequence according to any one of items 10-13.

Item 15. A virus having a genome comprising an ORF encoding the amino acid sequence according to any one of items 10-13.

Item 16. An amino acid sequence comprising SEQ ID NO: 5.

Item 17. A virus having a genome comprising an ORF encoding the amino acid sequence according to item 16.

Item 18. The virus according to any one of items 9, 15, 17, which is a PEDV.

Item 19. A PEDV comprising ORF-2 and ORF 3, with a proviso that the virus comprises a first deletion in said ORF2/ORF3, wherein said first deletion is a deletion of SEQ ID NO: 6 or a deletion of a nucleic acid sequence comprising SEQ ID NO: 6, with a proviso that said virus expresses amino acid sequence comprising SEQ ID NO: 3 or a sequence that is at least 90% identical thereto, with a further proviso that C-terminal amino acids of said SEQ ID NO: 3 is QPLAL (SEQ ID NO: 4).

Item 20. The PEDV of item 20, which further comprises a second deletion in said ORF-3, wherein said second deletion is a deletion of SEQ ID NO: 7 or a deletion of a nucleic acid sequence comprising SEQ ID NO: 7.

Item 21. The PEDV of item 19 or 20, wherein said virus comprises wild-type ORFs encoding E, M, and N proteins.

Item 22. The PEDV according to any one of items 19-21 wherein the first deletion and the second deletion are distinct.

Item 23. The PEDV according to any one of items 18-22 which lacks a functional protein expressed by ORF-3.

Item 24. The PEDV according to any one of items 18-23 which has a genome according to SEQ ID NO: 10 or a sequence that is at least 90% identical thereto.

Item 25. The PEDV according to any one of items 18-24 which is derived from a PEDV strain selected from the group consisting of strains DJ, AJ1102, CH/ZJCS03/2012, CH/JXZS03/2014, CH/JXFX01/2014, CH/JXJJ08/2015, CH/JXGZ04/2015, CH/JXJA89/2015, CH/JXDX119/2016, CH/JXJGS11/2016, CH/JXWN13/2016, CH/JXJJ18/2017, CH/JXNC38/2017, CH/JX/01, CH/JX-1/2013, CH/JX-2/2013, AH2012, GD-B, BJ-2011-1, CH/FJND-3/2011, AJ1102, GD-A, CH/GDGZ/2012, CH/ZJCX-1/2012, CH/FJZZ-9/2012.

Item 26. The PEDV according to items 18-24, which is derived from PEDV strain DJ.

Item 27. A further attenuated PEDV which is a progeny of the parental PEDV of claim 24.

Item 28. The further attenuated PEDV according to claim 27, wherein said parental PEDV has a genome according to SEQ ID NO: 10.

Item 29. A vaccine comprising the PEDV according to any one of items 18-26 or a further attenuated PEDV according to item 27 or 28.

Item 30. The vaccine according to item 29, wherein the PEDV according to any one of items 18-26 is attenuated.

Item 31. A method of preventing a swine animal from PEDV infection comprising administering to said swine the vaccine according to item 29 or 30.

Item 32. The method according to item 31, wherein said vaccine is administered orally.

Item 33. The method of item 31 or 32, wherein said swine animal is a sow, wherein said vaccine is administered a first time about 28-42 days before the farrowing and wherein further said vaccine is administered a second time about 7-21 days before the farrowing.

Item 34. A method of protecting a piglet from PEDV infection comprising administering to said piglet colostrum from a sow vaccinated with the vaccine according to item 29.

Item 35. The method according to item 34, wherein said first vaccination and/or said second vaccination is oral.

Item 36. The method according to item 34 or 35, wherein said piglet is at least 3 days old.

Item 37. The method according to item 36, wherein said piglet is at least five days old.

Item 38. The method according to any one of items 34-37 wherein said sow was vaccinated about 35 days from farrowing.

Item 39. The method according to any one of items 34-38 wherein said sow was vaccinated about 14 days from farrowing.

The following examples are presented as illustrative embodiments but should not be taken as limiting the scope of the invention. Many changes, variations, modifications, and other uses and applications of this invention will be apparent to those skilled in the art.

### EXAMPLES

### Example 1—Safety of the PEDV Vaccine

A virulent pandemic PEDV strain from the south of China, PEDV-DJ, which belongs to the G2a group, was serially propagated in Vero cells for up to 57 passages. The ORF2-ORF3 region of the virus of different passages was sequenced to monitor the virulence-associated mutations and genetic stability. The primers used in the sequencing were:
ORF3 24655-F: 5′-TCA TTA CTA GTG TTC TGC TGC ATT TC-3′ (SEQ ID NO: 11);
ORF3 25541-R: 5′-CAC AGA TTA ACC AAT TGG ACG AAG GT-3′ (SEQ ID NO: 12);

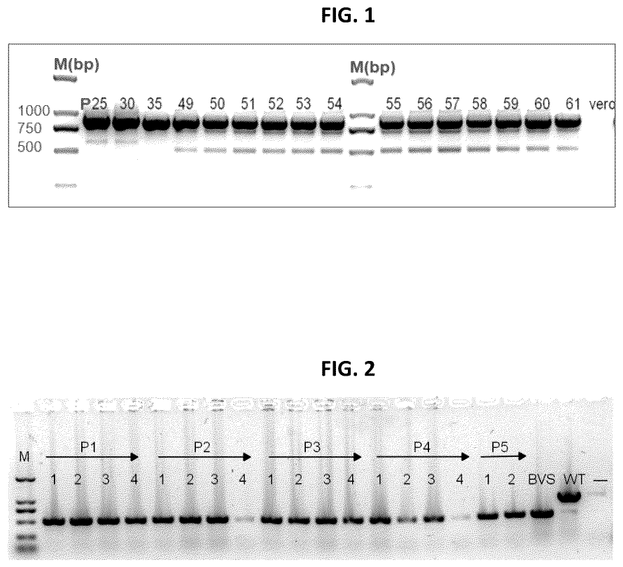

The electrophoretic map of continuously passaged virus is provided in FIG. 1. ORF2-ORF3 region of different passages of cell-adapted PEDV strains. Mutant with a large fragment deletion in ORF2-ORF3 region was identified at P49 (arrows).

To verify the safety of the vaccines strain, suckling piglets of 5-7 days of age were orally inoculated with PEDV vaccine strain (10⁷ TCID₅₀/pig). Clinical signs (overall behavior, appetite), particularly the clinical manifestations of the digestive system, were evaluated. Fecal morphology was scored, and the number of dead/alive piglets post-inoculation was counted.

Both groups of piglets survived. There were no remarkable visible differences in small intestines of piglets vaccinated with the PEDV according to the invention and non-vaccinated piglets. In contrast, small intestines of piglets inoculated with the virulent strain of PEDV were dilated with accumulated yellow fluid and had thin transparent walls as a result of villous atrophy.

Fecal consistency scores were evaluated according to the following criteria: 1-normal feces (solid); 2-pasty; semi-solid; 3-yellowish watery. No differences were found between fecal consistency scores of non-vaccinated piglets and in piglets vaccinated with the PEDV strain of the invention.

### Example 2—Non-Reversion to Virulence

Suckling piglets of 5-7 days of age were orally inoculated with 10⁷ TCID₅₀ of PEDV vaccine strain. Clinical signs (overall mental state, appetite), particularly the clinical manifestations of the digestive system, were evaluated. Fecal morphology was scored, and the number of dead/alive animals was counted to comprehensively evaluate the safety of the strain. Infected piglets were euthanized for the 3 days post-inoculation. Small intestine as well as content of the small intestine were collected and used to as antigen to inoculate the next round of piglets. This inoculation pattern was repeated 5 times to complete the reversion to virulence study. Clinical samples from each round of inoculation were used for the isolation of vaccine strain, and the region of the genetic marker of the vaccine strain was sequenced for each round of inoculation. Two criteria, namely the genetic stability and the morbidity of the piglets, were used to evaluate the virulence of the vaccine strain.

FIG. 2 is an electrophoretic map of nucleic acid of 5 continuously passaged virus, demonstrating that the virus is genetically stable. All piglets were alive at the end of the experiment.

These animal studies revealed that the virulence of the vaccine strain has been remarkably reduced based on the clinical signs and the survival rate of piglets for each passage, meanwhile, the genetic marker in ORF2-ORF3 regions remain stable after 5 passages in piglets. Conclusively, the PEDV vaccine strain is stable in both phenotype and genotype.

### Example 3—Immunogenicity

Six naïve sows were orally immunized and boosted at 60-day and 14-day prior to farrowing, respectively. Each dose of the vaccine contained 1×10⁵ TCID₅₀/ml of the virus according to Example 1. Piglets from sows were orally challenged at 5-7 days of age with PEDV TM strain, 10² TCID₅₀/ml, and clinical symptoms and death/survival were observed for 10 days after challenge.

In addition, serum samples from sows (on the first immunization day and farrowing) and suckling piglets (at 5-7 days of age and 10-day post challenge) were collected and evaluated via ELISA (IDEXX PEDV-IgA ELISA kit) and SN assay (Self-established assay, validated).

The survival rate of the piglets from immunized sows were 100%, while none of piglets from the negative control survived after challenge. Immunized sows and piglets after challenge were clean and energetic and suckled the milk proactively, with no diarrhea clinical signs.

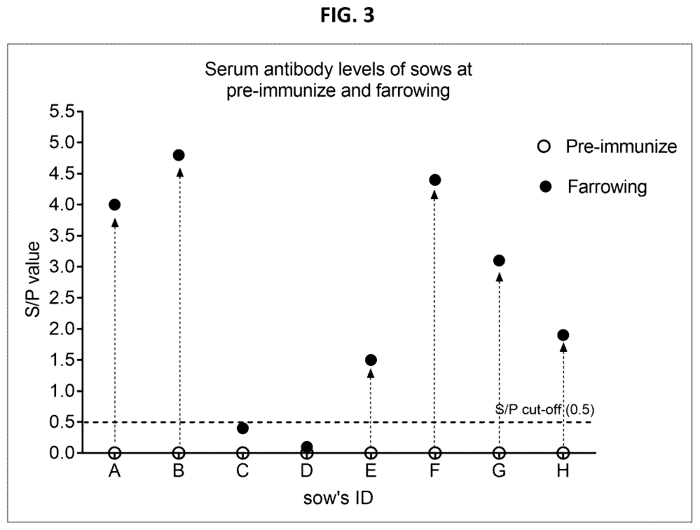

FIG. 3 shows PEDV antibody levels of sows in pre-immunize day and farrowing day (14 days after second immunization). Sow C was excluded from further analysis due to immunization failure, Sow D is a control sow orally immunized with PBS only instead of PEDV antigen. In the remaining six sows, the antibody levels reached protective titer.

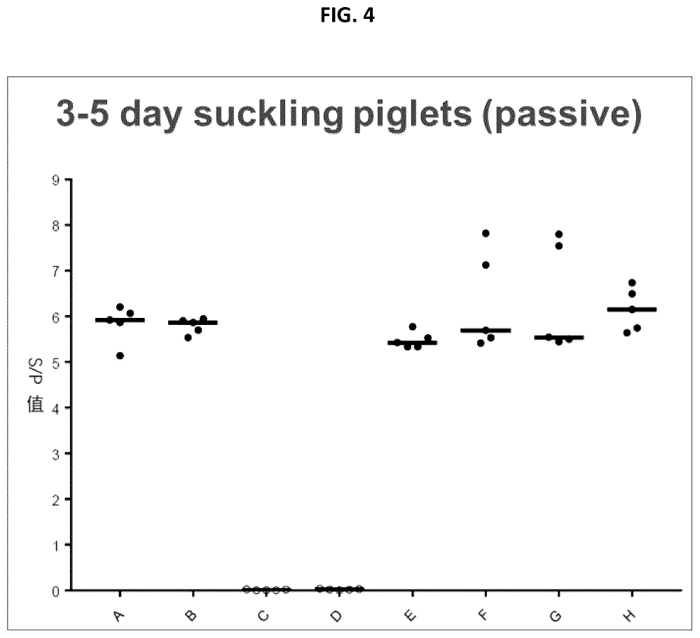

FIG. 4 shows antibody levels in 3-5 day old suckling piglets. These are maternal antibodies transferred to the piglets with colostrum of the mother previously vaccinated as described above.

Taken together, these data show that oral administration of the virus according to the invention to pre-farrowing sows is sufficient to transfer protective immunity to suckling piglets to protects said piglets against the challenge with a virulent strain PEDV.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-18. (canceled)

19. A PEDV comprising ORF-2 and ORF 3, with a proviso that the virus comprises a first deletion in said ORF2/ORF3, wherein said first deletion is a deletion of SEQ ID NO: 6 or a deletion of a nucleic acid sequence comprising SEQ ID NO: 6, with a proviso that said virus expresses amino acid sequence comprising SEQ ID NO: 3 or a sequence that is at least 90% identical thereto, with a further proviso that C-terminal amino acids of said SEQ ID NO: 3 is QPLAL (SEQ ID NO: 4).

20. The PEDV of claim 20, which further comprises a second deletion in said ORF-3, wherein said second deletion is a deletion of SEQ ID NO: 7 or a deletion of a nucleic acid sequence comprising SEQ ID NO: 7.

21. The PEDV of claim 19, wherein said virus comprises wild-type ORFs encoding E, M, and N proteins.

22. The PEDV according to claim 19 wherein the first deletion and the second deletion are distinct.

23. The PEDV according to claim 19 which lacks a functional protein expressed by ORF-3.

24. The PEDV according to claim 19 which has a genome according to SEQ ID NO: 10 or a sequence that is at least 90% identical thereto.

25. The PEDV according to claim 19 which is derived from a PEDV strain selected from the group consisting of strains DJ, AJ1102, CH/ZJCS03/2012, CH/JXZS03/2014, CH/JXFX01/2014, CH/JXJJ08/2015, CH/JXGZ04/2015, CH/JXJA89/2015, CH/JXDX119/2016, CH/JXJGS11/2016, CH/JXWN13/2016, CH/JXJJ18/2017, CH/JXNC38/2017, CH/JX/01, CH/JX-1/2013, CH/JX-2/2013, AH2012, GD-B, BJ-2011-1, CH/FJND-3/2011, AJ1102, GD-A, CH/GDGZ/2012, CH/ZJCX-1/2012, CH/FJZZ-9/2012.

26. The PEDV according claim 19, which is derived from PED strain DJ.

27. A further attenuated PEDV which is a progeny of the parental PEDV of claim 24.

28. The further attenuated PEDV according to claim 27, wherein said parental PEDV has a genome according to SEQ ID NO: 10.

29. A vaccine comprising the PEDV according to claim 19.

30. The vaccine according to claim 29, wherein the PEDV is attenuated.

31. A method of preventing a swine animal from PEDV infection comprising administering to said swine the vaccine according to claim 29.

32. The method according to claim 31, wherein said vaccine is administered orally.

33. The method of claim 31, wherein said swine animal is a sow, wherein said vaccine is administered a first time about 28-42 days before the farrowing and wherein further said vaccine is administered a second time about 7-21 days before the farrowing.

34. A method of protecting a piglet from PEDV infection comprising administering to said piglet colostrum from a sow vaccinated with the vaccine according to claim 29.

35. The method according to claim 34, wherein said first vaccination and/or said second vaccination is oral.

36. The method according to claim 34, wherein said piglet is at least 3 days old.

37. The method according to claim 36, wherein said piglet is at least five days old.

38. The method according to claim 34 wherein said sow was vaccinated about 35 days from farrowing.

39. The method according to claim 34 wherein said sow was vaccinated about 14 days from farrowing.
